(19) United States
(12) Patent Application Publication
(10) Pub. No.: US 2024/0268982 A1
(43) Pub. Date: Aug. 15, 2024

(54) MEDICAL AID OR SPORTS AID COMPRISING A BOWDEN CABLE

(71) Applicant: BAUERFEIND AG, Zeulenroda-Triebes (DE)

(72) Inventors: Gerald Stier, Zeulenroda-Triebes (DE); Sandro Hebenstreit, Zeulenroda-Triebes (DE); Hans B. Bauerfeind, Zeulenroda-Triebes (DE)

(21) Appl. No.: 18/567,764

(22) PCT Filed: Jun. 1, 2022
(86) PCT No.: PCT/EP2022/064865

§ 371 Date: Dec. 6, 2023

(30) Foreign Application Priority Data
Jun. 7, 2021 (DE) 10 2021 205 698.9

(51) Int. Cl. A61F 5/01

(52) U.S. Cl. CPC A61F 5/0123; A61F 2005/0165

(57) ABSTRACT

The present invention relates to a medical aid or sports aid (100), comprising a Bowden cable (10, 40), wherein the Bowden cable (10, 40) comprises a core (11, 41), wherein the core (11, 41) runs at least in a partial region in a casing (12, 42), wherein the casing (12, 42) is stretchable at least in a partial region, and the use of a Bowden cable (10, 40), having a core (11, 41) for tensioning a medical aid or a sports aid (100), wherein the core (11, 41) runs at least partially displaceably in a casing (12, 42) and the casing (12, 42) is stretchable.

### CROSS REFERENCE TO RELATED APPLICATIONS

This application is a U.S. a national stage application under 35 U.S.C. 371 of PCT/EP2022/064865 (“the '865 PCT application”), filed Jun. 1, 2022, which claims priority to German patent application DE 10 2021 205 698.9, filed Jun. 7, 2021 (“the '698.9 Priority Application”). The contents of the '865 PCT Application and '698.9 Priority application are incorporated herein in their entireties.

### TECHNICAL FIELD

The present invention relates to a medical aid or sports aid comprising a Bowden cable, wherein the Bowden cable comprises a core, wherein the core runs at least in a partial region in a casing, wherein the casing is stretchable at least in a partial region and the use of a Bowden cable having a core for tensioning a medical aid or a sports aid, wherein the core runs at least partially displaceably in a casing and the casing is stretchable.

### SUMMARY

A Bowden cable is a movable machine element for transmitting a mechanical movement as well as pressure and tensile forces by means of a flexible combination of a rope, in particular a wire rope, and a housing or casing that is pressure-resistant in the direction of travel. A Bowden cable usually consists of a tension rope, also known as a core, for example a steel wire or wire rope, which is displaceably laid in a flexible but pressure-resistant casing, also known as a housing. The casing serves to guide the cable and as a counter bearing to support the tensile forces to be transmitted. A Bowden cable can therefore transmit tensile forces along any curved path as long as the radius does not fall below a certain value. Bowden cables are used in a wide variety of technical areas, but particularly in vehicle construction. In medical technology, the use of Bowden cables in joint orthoses is described in EP 1 186 279 A1 and DE 10 2009 003 999 A1.

In medical aids or sports aids, Bowden cables are usually replaced by simple cable pulls, in which ropes or cords are tensioned, for example by rolling them up. The ropes or cords can run freely or they are guided in laminations or knitted channels.

A disadvantageous feature of such ropes is that they rub against the medical aid or sports aid, for example against a knitted fabric, during movement and thus damage the knitted fabric in the long term, and can also cut into the skin of the person wearing the aid.

The technical problem underlying the present invention is the provision of tensioning means for medical aids or sports aids which avoid the problems of the prior art. In particular, the technical problem of the present invention is the provision of alternatives to cable pulls in medical aids or sports aids, in particular when used as tensioning means, which cause less or no friction when moving the cable pull, in particular the tensioning means, in particular in deflection areas and other areas where tensioning means often tend to clamp.

The present invention solves the underlying technical problem by means of a medical aid or sports aid comprising a Bowden cable, wherein the Bowden cable comprises a core, wherein the core runs at least in a partial region in a casing, wherein the casing is stretchable at least in a partial region.

Surprisingly, it has been shown that Bowden cables can be used in an advantageous manner instead of cable pulls in medical aids, in particular orthopaedic aids, and sports aids, in particular as tensioning elements, if the casing, i.e. the housing, of the Bowden cables is stretchable at least in a partial region. With conventional Bowden cables and a conventional use of these, the casing does not have to be stretchable, but only bendable, as the core is only slidable in the casing and therefore no shortening or lengthening of the casing is necessary. However, this is necessary when the core is used as a tensioning element in a medical aid or sports aid, meaning that a conventional Bowden cable could not be used here. The design of at least a partial region of the casing as a stretchable casing now advantageously leads the Bowden cable as a whole to be flexible in length and thus can also be used as a tensioning element in a medical aid or sports aid.

Various possibilities are known to the skilled person for making at least a partial region of the casing or the casing itself stretchable. In particular, the casing can be made stretchable by means of slits, for example by means of a spiral-shaped slit. Alternatively, the casing of the Bowden cable can also be accordion-shaped, at least in partial regions, or be made of a stretchable plastic or rubber.

In a preferred embodiment, the casing is made of plastic.

In a preferred embodiment, the casing has at least one slit.

In a preferred embodiment, the casing is spiral-shaped at least in a partial region.

The casing can be slitted throughout or only in partial regions. The slitting can be either annular, i.e. in particular spiral-shaped, or meander-shaped. The slits in the casing can also be non-annular. For example, the slits can be offset, parallel or inclined.

A casing with at least one slit, in particular a spiral-shaped slit or the design of an inherently spiral-shaped casing, has the advantage that at least a partial region of the casing, but also the entire casing, can be designed to be stretchable and at the same time the core in the casing can still be displaced with low friction. This allows the coefficient of friction to be advantageously distributed between the core and the casing.

In a preferred embodiment, the casing is elastic in at least a partial region.

In a preferred embodiment, the casing has a round cross-section or a stadium-shaped cross-section. The cross-section of the casing, in particular the cross-section of the outer surface of the casing, can also be of a different shape, for example angular, in particular polygonal, or oval.

The casing can therefore have a round cross-section, for example, as is common for Bowden cables from the prior art. Alternatively, the casing can also have a stadium-shaped cross-section, at least in partial regions. A stadium-shaped cross-section is a cross-section in which two parallel straight lines are connected to each other by two arches. The casing with a stadium-shaped cross-section is then designed in particular as an internally hollow flat strip. This allows the casing to rest in an advantageous manner with one of the two flat, straight surfaces on the medical aid or sports aid. As a result, the flat band, especially if the Bowden cable is designed as a tensioning element, does not cut so much into the user's skin under the Bowden cable.

The core can be designed as a conventional core, for example as a wire or rope. The core can be made of metal, plastic or other materials. However, the core can also be designed as a flat band, for example if it runs in a casing designed as a flat band.

In a preferred embodiment, the Bowden cable is designed as a tensioning element.

In a preferred embodiment, the Bowden cable is associated with a tensioning system.

In a preferred embodiment, the core of the Bowden cable can be tensioned via a tension regulating element. In a preferred embodiment, the core of the Bowden cable can be rolled onto the tension regulating element.

Various tensioning elements and tension regulating elements such as rollers in medical aids, for example orthopaedic aids, in particular orthoses, or sports aids, in which the tensioning element, usually a rope or a cord, can be replaced by a Bowden cable according to the invention, are known to those skilled in the art. The Bowden cable according to the invention can also be used in an advantageous manner as a tensioning element in the construction of new medical aids or sports aids.

When tensioning the tensioning element, the core of the Bowden cable is tensioned, for example by winding or pulling, and then fixed. Accordingly, relaxation of the core takes place in the opposite way. By tensioning the core, for example, the medical aid or sports aid can be pressed more firmly against the wearer's body. In an advantageous way, the use of the Bowden cable now guides the core to run with low friction in the casing and therefore does not rub or clamp on the medical aid or the sports aid. Furthermore, the casing increases the contact surface so that the Bowden cable cuts into the wearer's skin less than a conventional rope. Due to the stretchability of the casing, it can adapt to the length of the core running inside it in an advantageous manner during the tensioning or relaxation process.

Preferably, the medical aid or sports aid comprises a base body, for example made from a knitted fabric. Preferably, the base body is flexible and/or stretchable. However, the base body can also be a framework or similar. Preferably, the Bowden cable runs at least in partial regions on the base body.

In a preferred embodiment, the casing is connected to the medical aid or sports aid at its end regions. In a preferred embodiment, the casing is connected to the medical aid or sports aid only at its end regions. Alternatively or additionally, the casing may also be connected to the medical aid or sports aid at other sections or regions of the casing. Preferably, the connection is a fixed, non-displaceable connection, particularly at the end regions. On the other hand, connections can also be provided in which the casing is displaceably mounted on the medical aid or sports aid, for example in the case of guide-throughs and/or deflecting guides.

Like a conventional Bowden cable in a conventional use, the casing of the Bowden cable in the present invention can advantageously also be connected only by its end regions to the device assigned to it, in the present case to the medical aid or sports aid. This advantageously leads to the longitudinal course of the Bowden cable being displaceably mounted on the medical aid or sports aid and thus being able to adapt to the course of the wearer's body when the medical aid or sports aid is worn and, when the tensioning element is tensioned or relaxed, the casing of the Bowden cable can also run freely on the surface of the medical aid or sports aid and thus does not lead to the formation of creases.

Guide-throughs can also be provided in which the casing of the Bowden cable is displaceably mounted and which are preferably located on the medical aid or sports aid, in particular on the base body of the medical aid or sports aid. These guide-throughs can advantageously position the Bowden cable on the medical aid or sports aid without the Bowden cable being fixedly connected to the medical aid or sports aid in this region. The guide-throughs can, for example, be designed as a guide-through element, such as a pipe fastened to the medical aid or sports aid, through which the Bowden cable runs, in particular runs movably, i.e. the casing is also mounted displaceably in the pipe. As an alternative to a pipe, a channel can also be provided.

The guide-throughs can also be rotatably mounted on the medical aid or sports aid, in particular on the base body of the medical aid or sports aid, so that they can adapt to a change in the course of the Bowden cable by rotating accordingly.

The guide-throughs can also be designed as a deflecting guide.

In a preferred embodiment, the Bowden cable is guided by at least one deflecting guide, wherein the casing is preferably displaceably mounted in the deflecting guide.

The Bowden cable can be in an advantageous manner guided through deflecting guides in order to determine the course of the Bowden cable on the medical aid or sports aid. In a preferred manner, the Bowden cable is mounted in the deflecting guide in such a way that the casing is displaceable, so that the deflecting guide does not impair the overall displaceability of the casing and the Bowden cable in relation to the medical aid or sports aid in an advantageous manner.

The deflecting guides can also be rotatably mounted on the medical aid or sports aid, in particular on the base body of the medical aid or sports aid.

In a preferred embodiment, the medical aid or sports aid is worn on the body.

Preferably, the medical aid or sports aid is therefore not a device in which conventional Bowden cables are usually used, for example for brakes on rollators.

Preferably, the medical aid or sports aid is not a device in which limbs, for example fingers, are moved by a Bowden cable.

Preferably, the medical aid is not a surgical aid.

Preferably, the medical aid is an orthopaedic aid.

In a preferred embodiment, the medical aid or the sports aid is a bandage or an orthosis.

Preferably, the medical aid or sports aid is not a prosthesis, for example an artificial hand.

Preferred orthoses according to the invention act externally directly on the body. They serve to stabilise, relieve, immobilise or mobilise limbs or the trunk or to correct incorrect postures. They can also replace partially lost functions or prevent deformities. Prostheses preferably not comprised by the invention completely or partially replace missing or damaged body parts such as limbs, ears and nose as well as organs or organ parts. They create a functional compensation and produce the optical appearance again.

The present invention also solves the underlying technical problem by using a Bowden cable having a core for tensioning a medical aid or a sports aid, wherein the core runs at least partially displaceably in a casing and the casing is stretchable.

Preferred embodiments of the Bowden cable used result from the description for the medical aid or sports aid according to the invention comprising such a Bowden cable.

Preferred embodiments of the invention are shown in the sub-claims. Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

The present invention is explained in more detail with reference to the following figures and examples, without these being to be understood as limiting. It shows:

FIGS. 1a and 1b A Bowden cable of a medical aid or sports aid according to the invention in an unstretched (la) and stretched or tensioned (1b) state;

FIGS. 2a and 2b an alternative embodiment of the Bowden cable in an unstretched (2a) and stretched or tensioned (2b) state;

FIG. 3 the Bowden cable as a tensioning system with a tension regulating element;

FIG. 4 a Bowden cable with a guide-through element on a medical aid or sports aid;

FIG. 5 sectional view of the Bowden cable of FIG. 4;

FIG. 6 a Bowden cable in a deflecting guide;

FIG. 7 a medical aid or sports aid according to the invention with a Bowden cable as a tensioning system; and

FIGS. 8a-8c further alternative embodiments of the Bowden cable.

### DETAILED DESCRIPTION

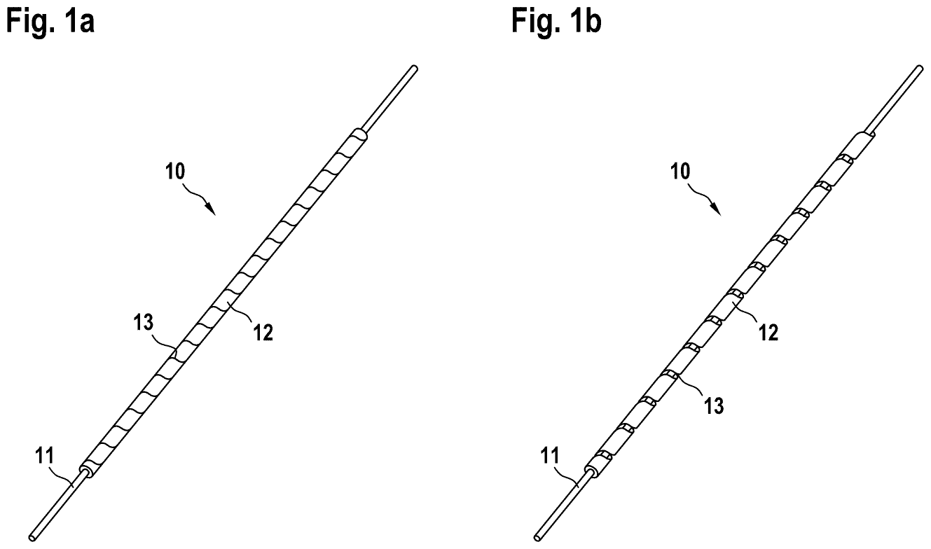

FIG. 1 shows a Bowden cable (10) of a medical aid or sports aid according to the invention. The Bowden cable (10) comprises a core (11), for example made of plastic or metal, and a casing (12). The casing (12) has a throughout slit (13) whereby it is stretchable. FIG. 1a shows the casing (12) in an unstretched state, FIG. 1b shows the casing (12) in a stretched or tensioned state. The slit (13) is correspondingly wider.

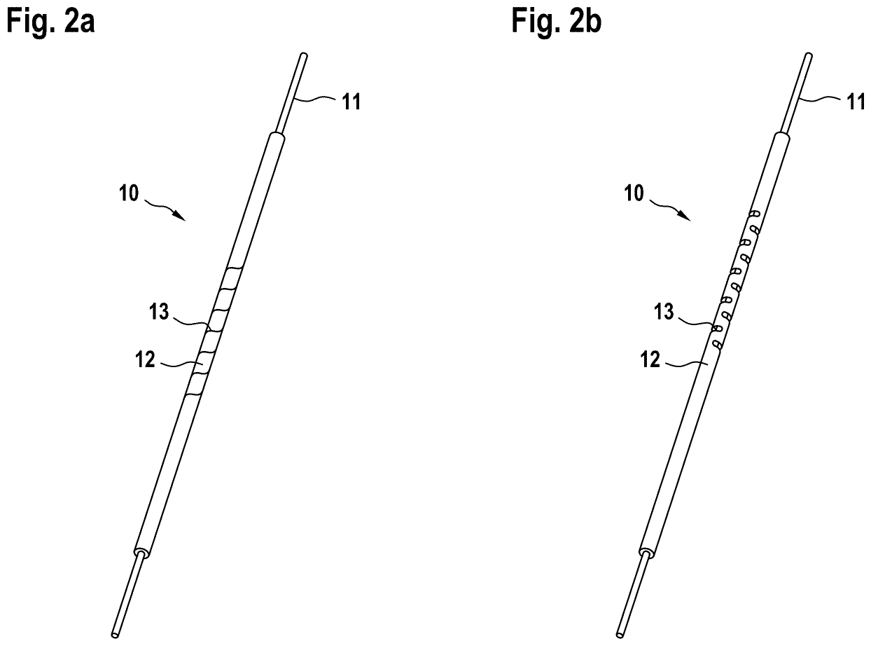

FIG. 2 shows two alternative embodiments of such a Bowden cable (10). The core (11) of the Bowden cable (10) is the same in FIGS. 2a and 2b. In FIG. 2a, the casing (12) has a stretchable region in which a slit (13) runs in an annular or spiral shape. In FIG. 2b, a plurality of slits (13) arranged in a meander-shaped pattern are present in the stretchable region of the casing (12). Both embodiments of the slits (13) allow the casing (12) to be stretchable or tensionable in the region of the slits (13).

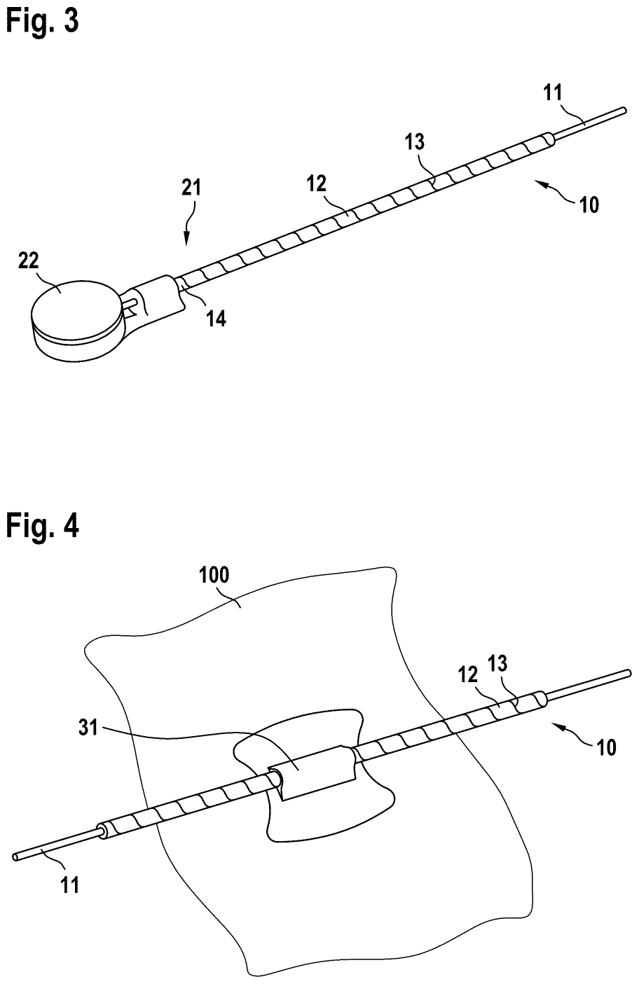

FIG. 3 shows the Bowden cable (10) with a core (11) and a casing (12) that is stretchable through a spiral-shaped slit (13) and is used as a tensioning element (21). This is achieved by the fact that the core (11) can be rolled onto a tension regulating element (22). During tensioning, the core (11) slides in the casing (12), wherein the casing (12) is connected to the tension regulating element (22) only at its end region (14) and is thus connected to the medical aid or sports aid located on the tension regulating element (22) only at this end region (14). During relaxation, the core (11) is unrolled from the tension regulating element (22) so that it becomes longer. Due to the stretchability of the casing (12), i.e. its spiral-shaped slit (13), the casing can stretch and thus also completely encase the extended core (11), so that the core (11) is not exposed but continues to be completely shielded by the casing (12).

FIG. 4 shows a section of a medical aid or sports aid (100) according to the invention, to which a Bowden cable (10) with a core (11) and a casing (12) stretchable due to a spiral-shaped slit (13) is assigned. In the area shown, the Bowden cable (10) is not fixedly connected to the carrier material of the medical aid or sports aid (100), but can slide on it. The Bowden cable (10) is guided in a slidable manner through a tubular guide-through in the form of a guide-through element (31). Not only the core (11) of the Bowden cable (10) can slide through the guide-through element (31), but also its casing (12). Thus, the guide-through element (31) does not serve to fix the Bowden cable (10) of the medical aid or sports aid (100), but only to position it, wherein the Bowden cable including the casing remains displaceable and stretchable in the longitudinal direction.

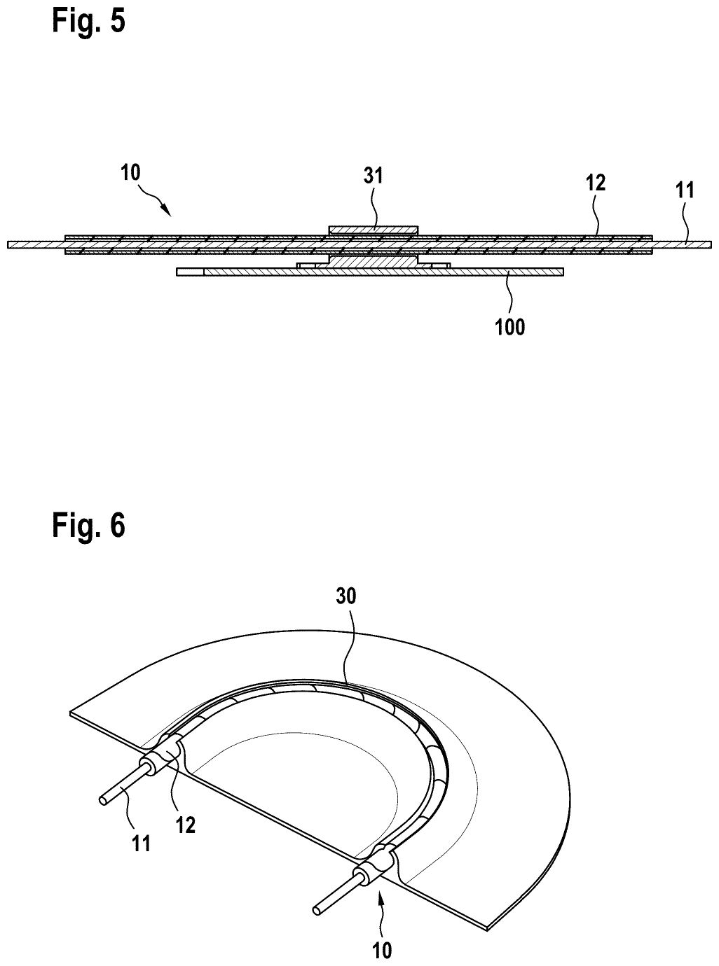

FIG. 5 shows a side view of the cross-section of the structure shown in FIG. 4. The Bowden cable (10) with core (11) and stretchable casing (12) can be seen, wherein the Bowden cable (10) is mounted displaceably in the guide-through element (31) so that it is positioned on the carrier material of the medical aid or sports aid (100), without the Bowden cable (10) being fixedly connected there to the carrier material (100), since the entire Bowden cable (10) including the casing (12) is displaceably mounted in the guide-through element (31).

FIG. 6 shows a Bowden cable (10) in a guide-through, which is designed as a deflecting element or deflecting guide (30). Here too, the casing (12) of the Bowden cable (10) is displaceably mounted in the channel-shaped deflecting guide (30) so that, on the one hand, the entire Bowden cable (10) with core (11) and casing (12) is displaceably mounted in the deflecting guide (30) and, on the other hand, the casing (12) also remains stretchable in the deflecting guide (30). The deflecting guide (30) can be fastened in a simple manner to a carrier material of a medical aid or sports aid, for example by gluing, sewing or welding.

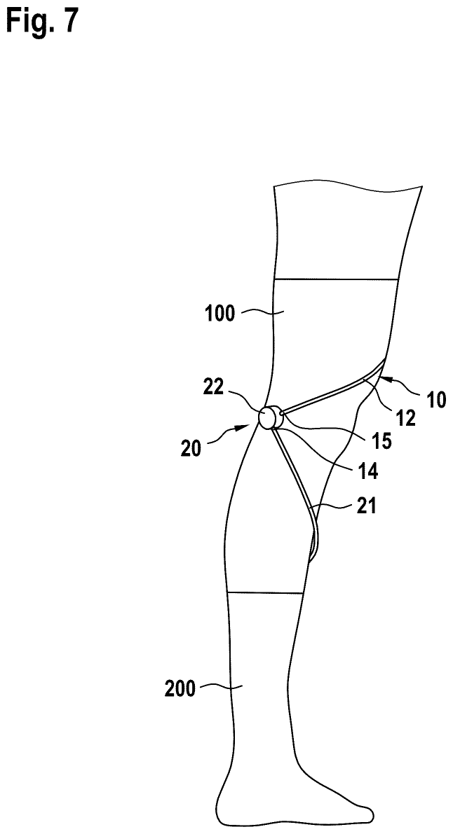

FIG. 7 shows an exemplary embodiment of a medical aid or sports aid (100) that is worn on a body, in this case a leg (200). The medical aid or sports aid (100) has a tensioning system (20) comprising a Bowden cable (10) with a casing (12) that is stretchable by slitting and a core that runs in the casing and is therefore not visible as a tensioning element (21) and a tension regulating element (22). The tension regulating element (22) can be used to roll up the core of the Bowden cable (10) and thus generate tension in the tensioning element (21). The stretchable casing (12) is only fixedly connected at its ends (14, 15) to the tension regulating element (22) and thus to the medical aid or sports aid (100) as a whole. As a result, the casing (12) can stretch when the core of the tensioning element (21) is tensioned or relaxed and thereby slide on the carrier material of the medical aid or sports aid (100). On the one hand, this makes it possible for the casing (12) to cover the core completely even when it is stretched and tensioned, so that the core does not rub on the carrier material of the medical aid or sports aid (100) and cannot damage it, and on the other hand, it is made possible for the casing (12) of the Bowden cable (10) to move freely on the carrier material (100), thus enabling suitable positioning and a suitable course of the tensioning element (21). If necessary, it is possible to influence this positioning by means of guide-through elements or deflection elements in which the entire Bowden cable (10) with casing (12) is displaceably mounted.

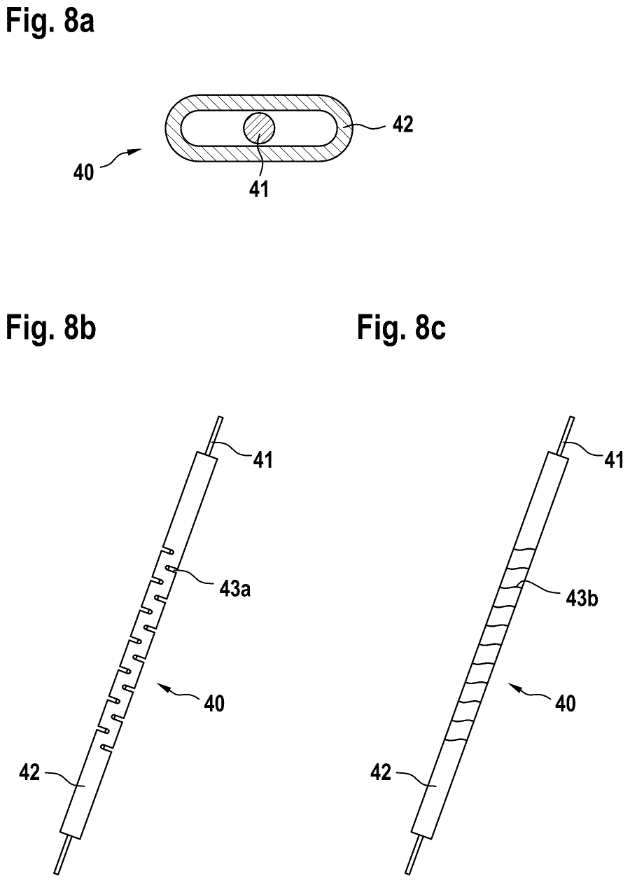

FIG. 8 shows a further embodiment of a Bowden cable (40) according to the invention with a core (41) and a casing (42). In this embodiment, the casing (42) has a stadium-shaped cross-section, i.e. has two parallel straight sections that are connected to each other by two arched sections. This cross-section of the casing (42) allows the core (41) to be mounted in the casing so that it is displaceably not only in the longitudinal direction, but also in the transverse direction. In addition, this design of the casing (42) as a flat band-like element allows the casing to rest with one of the flat sides on the medical aid or sports aid. As a result, the Bowden cable (40) does not cut so much into the user's skin lying under the Bowden cable (40) during tensioning, regardless of whether or not the base body of the medical aid or sports aid is still located between the Bowden cable (40) and the user's skin.

FIG. 8a shows the cross-section of the Bowden cable (40) with the stadium-shaped or flat band-like casing (42) and the core (41) located therein.

FIGS. 8b and 8c show a top view of such a Bowden cable (40) with core (41) and casing (42), wherein the casing (42) has different types of slits to produce stretchability. In FIG. 8b, the slits (43a) are meander-shaped. In FIG. 8c, one slit (43b) is annular, i.e. spiral-shaped. In both FIG. 8b and FIG. 8c, only a partial region of the casing (42) is provided with the slits (43a, 43b).

1. A medical aid or sports aid comprising a Bowden cable, wherein the Bowden cable comprises a core, wherein the core runs at least in a partial region in a casing, wherein the casing is stretchable at least in a partial region, wherein the Bowden cable is designed as a tensioning element, and the Bowden cable is guided through at least one guide-through element, wherein the casing is displaceably mounted in the guide-through element.

2. The medical aid or sports aid according to claim 1, wherein the casing has at least one slit.

3. The medical aid or sports aid according to claim 1, wherein the casing is spiral-shaped at least in a partial region.

4. The medical aid or sports aid according to claim 1, wherein the casing is elastic at least in a partial region.

5. The medical aid or sports aid according to claim 1, wherein the casing has at least in sections a round cross-section, an angular cross-section, an oval cross-section or a stadium-shaped cross-section.

6. The medical aid or sports aid according to claim 1, wherein the core is designed as a wire, a rope or a flat band.

7. The medical aid or sports aid according to claim 1, wherein the Bowden cable is associated with a tensioning system.

8. The medical aid or sports aid according to claim 1, wherein the core of the Bowden cable can be tensioned via a tension regulating element, such that the core of the Bowden cable can be rolled up onto the tension regulating element.

9. The medical aid or sports aid according to claim 1, wherein the casing is connected to the medical aid or sports aid at its end regions or in one or more sections.

10. The medical aid or sports aid according to claim 1, wherein the Bowden cable is guided through a deflecting guide, and wherein the casing is displaceably mounted in the at least one deflecting guide.

11. The medical aid or sports aid according to claim 1, wherein the medical aid or sports aid has a base body.

12. The medical aid or sports aid according to claim 1, wherein the medical aid or sports aid is worn on a user's body.

13. The medical aid or sports aid according to claim 1, wherein the medical aid or sports aid is a bandage or an orthosis.

14. A method of tensioning a medical aid or a sports aid using a Bowden cable, the method comprising:
selecting a Bowden cable having a core for tensioning a medical aid or a sports aid, wherein the core runs at least partially displaceably in a casing and the casing is stretchable, wherein the Bowden cable is guided through a guide-through element, and wherein the casing is displaceably mounted in the guide-through element.

15. The method of claim 14, wherein the method further comprises selecting the medical aid or the sports aid, and wherein the medical aid or the sports aid is an orthopaedic aid.

16. The method of claim 14, further comprising the step of tensioning the Bowden cable, and wherein the step of tensioning the Bowden cable causes the core to slides in the casing; and
wherein the casing is coupled to the medical or sports aid via a tension regulating element, and wherein the casing is connected to the tension regulating element only at an end region of the casing, and therefore the casing is connected to the medical aid or sports aid only at the end region of the casing.

17. The method of claim 16, further comprising the step of relaxing the Bowden cable, and wherein during relaxing, the core is unrolled from the tension regulating element and the casing completely encases the unrolled core.

18. A medical or sports aid comprising:
a Bowden cable for tensioning a medical and/or sports aid, the Bowden cable comprising a core for tensioning the medical and/or sports aid, the core running at least partially displaceably in a stretchable casing having a slit; and
a guide-through element to position the Bowden cable on the medical or sports aid, but not fix the Bowden cable to the medical or sports aid, allowing the stretchable casing to remain displaceable and stretchable in a longitudinal direction.

19. The Bowden cable of claim 18, wherein the casing comprises a stretchable region, and wherein the stretchable region comprises at least one slit.
